(19) United States
(12) Patent Application Publication
(10) Pub. No.: US 2022/0046877 A1
(43) Pub. Date: Feb. 17, 2022

(54) Artificial Plant Seed and Uses Thereof

(71) Applicant: Agriculture Victoria Services Pty Ltd, Bundoora (AU)

(72) Inventors: Noel Cogan, Macleod (AU); Ehab Ibrahim Ahmed Mohamaden, Williamstown (AU); German Carlos Spangenberg, Bundoora (AU)

(21) Appl. No.: 17/297,746

(22) PCT Filed: Dec. 18, 2019
(86) PCT No.: PCT/AU2019/051397

§ 371 Date: May 27, 2021

(30) Foreign Application Priority Data
Dec. 20, 2018 (AU) 2018904875

(51) Int. Cl. A01H 4/00; A01H 6/28; A01H 17/00

(52) U.S. Cl. CPC A01H 4/006; A01H 17/00; A01H 6/28

(57) ABSTRACT

The present invention relates generally to an artificial plant seed and to its use for plant propagation, in particular, for the propagation of cannabis plants.

### FILING DATA

This application claims priority from Australian Patent Application Number 2018904875 filed 20 Dec. 2018. The disclosure of this application is included herein by reference.

### FIELD

The present invention relates generally to an artificial plant seed and to its use for plant propagation, in particular, for the propagation of cannabis plants.

### BACKGROUND

The generation of medicinal products from cannabis plants such as Cannabis sativa relies on a uniform crop that has typically been derived from vegetative propagation of a single genetic source plant or strain.

Due to the dioecious outbreeding nature of cannabis plants, regulated by a sex determination system (X-Y based system with presence of the Y chromosome creating male plants), commercial production systems based on seed-derived plants and crops are not possible, without significant efforts around plant breeding, as well as chemical generation of hermaphrodite plants. As a result, vegetative propagation through cuttings is the preferred method of commercial plant generation and cultivation. However, the maintenance of mother plants from which to routinely generate cuttings is an ongoing requirement that introduces problems associated with plant health and longevity.

Whilst the asexual vegetative propagation of cannabis plants through cuttings is useful for strain preservation, including the maintenance of genetic traits, it nevertheless exposes the propagating material to pests and other disease-causing pathogens.

There remains, therefore, an urgent need for improved methods and material for propagating cannabis plant material that overcomes, or at least partly alleviates, one or more of the difficulties or deficiencies associated with current methods of propagation.

### SUMMARY

The present disclosure is predicated, at least in part, on the inventors' unexpected finding that isolated meristematic plant tissue derived from a cannabis plant can be encapsulated by a biocompatible polymer to produce an artificial plant seed capable of complete plant regeneration. Thus, in an aspect disclosed herein, there is provided an artificial seed comprising isolated meristematic plant tissue encapsulated by a biocompatible polymer, wherein the meristematic plant tissue is derived from a cannabis plant and sterilised prior to encapsulation.

The inventors have also surprisingly found that co-encapsulating the isolated meristematic plant tissue with endophytes, such as in a secondary layer of a biocompatible polymer, enables the endophytes to colonise the plants during the plant regeneration process. Thus, in an embodiment disclosed herein, the biocompatible polymer comprises an endophyte.

In another aspect disclosed herein, there is provided a method of producing an artificial plant seed of meristematic cannabis plant tissue encapsulated by a biocompatible polymer, the method comprising:
(a) isolating meristematic plant tissue from a cannabis plant;
(b) sterilizing the isolated meristematic plant tissue of (a);
(c) coating the sterilized meristematic plant tissue of (b) in a biocompatible polymer solution; and
exposing the polymer-coated meristematic plant tissue of (c) to a complexing agent to solidify the biocompatible polymer, thereby encapsulating the isolated meristematic cannabis plant tissue with the biocompatible polymer.

In an embodiment disclosed herein, the biocompatible polymer comprises an endophyte.

### BRIEF DESCRIPTION OF THE DRAWINGS

FIG. 1 shows a mature Cannabis plant maintained as mother stock as a source of cuttings for strain multiplication.

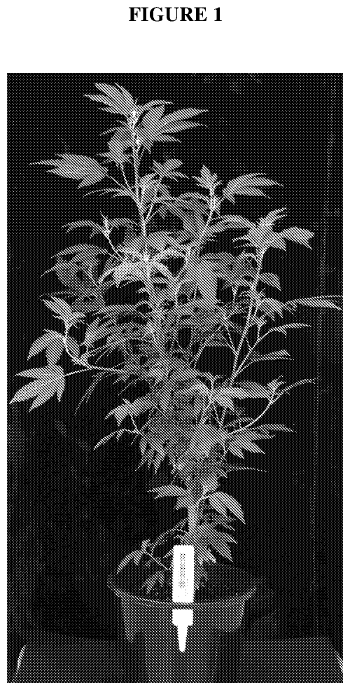

FIG. 2 shows auxiliary buds excised from the mother plant of FIG. 1.

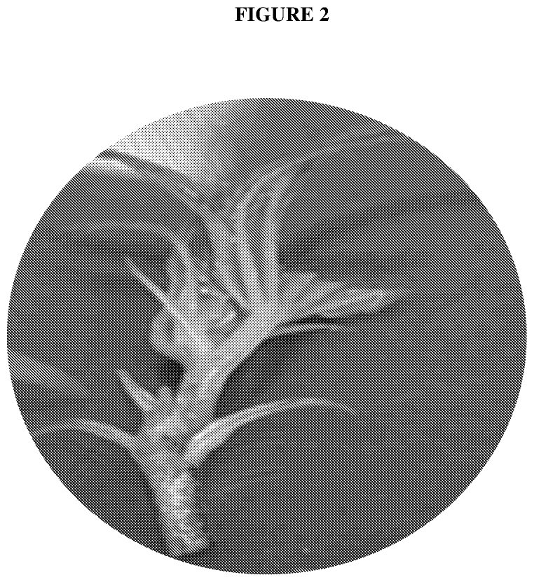

FIG. 3 shows artificial cannabis seeds (size approx. 3.0 mm).

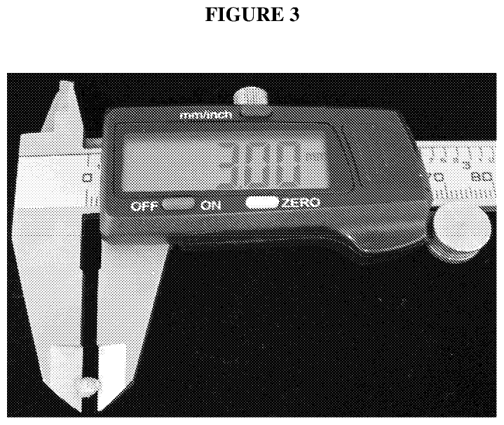

FIG. 4 shows sterilised auxiliary buds that have been trimmed to approx. 5 mm size segments.

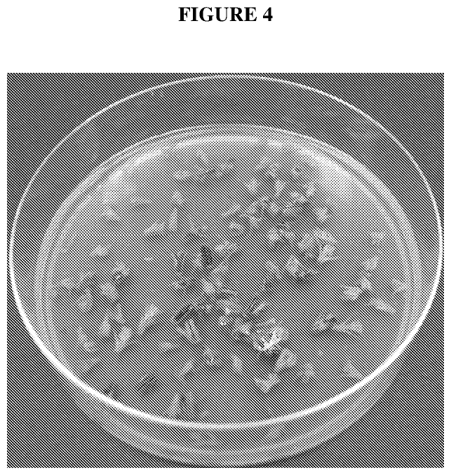

FIG. 5 shows the 5 mm segments of trimmed bud (from FIG. 4) immersed into a gel matrix.

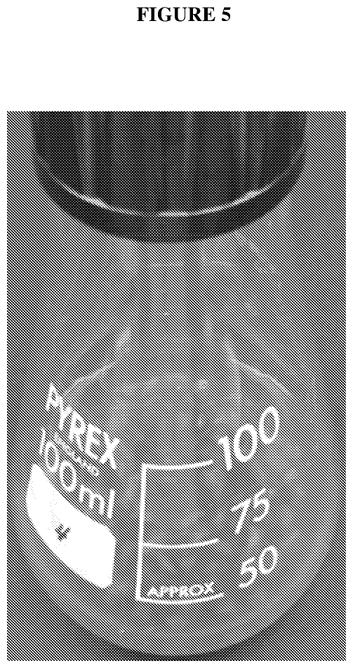

FIG. 6 shows gel encapsulated artificial seeds prepared for storage.

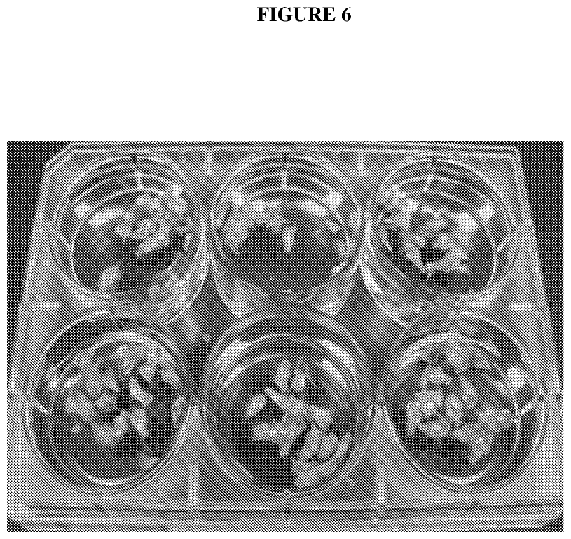

FIG. 7 shows the transfer of artificial seeds to regeneration medium with the progression of growth typically observed over a 7 day period to achieve shoot elongation, with 3 weeks required to generate a full plantlet.

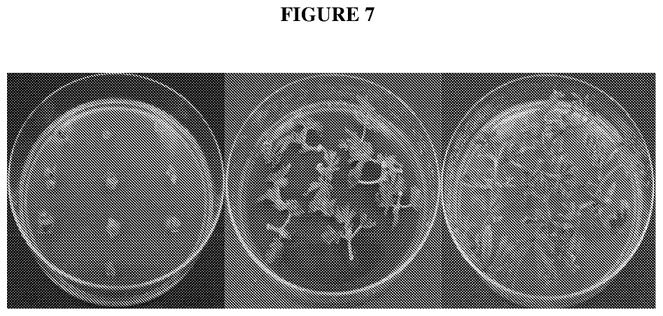

FIG. 8 shows a regenerating plantlet derived from a single artificial seed transferred to shooting/rooting medium at initial and mature stages depicted.

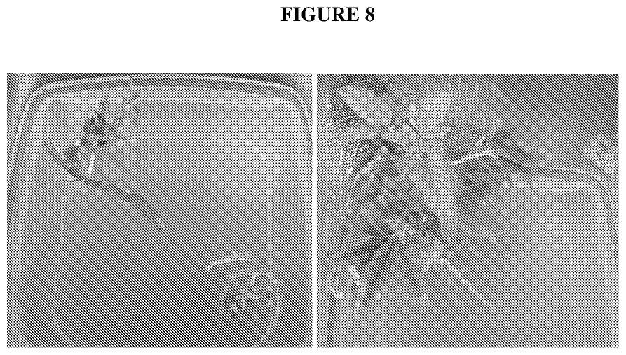

FIG. 9 shows a mixture of the bacterial strain cultures with sodium alginate. The three strains are labelled: Strain 1: Pantoea sp; Strain 2: Curtobacterium flaccumfaciens; Strain 3: Xanthomonas sp.

FIG. 10 shows formed microbe alginate beads for the three species; (A) Strain 1: Pantoea sp; (B) Strain 2: Curtobacterium flaccumfaciens; (C) Strain 3: Xanthomonas sp.).

FIG. 11 shows microbe growth post-1 week incubation directly inoculated onto medicinal cannabis regeneration medium (Table 3, shooting rooting medium) for the three microbial species; (A) Strain 1: Pantoea sp; (B) Strain 2: Curtobacterium flaccumfaciens; (C) Strain 3: Xanthomonas sp.).

FIG. 12 shows microbe growth from alginate bead post-1 week incubation on medicinal cannabis regeneration medium (Table 3, shooting rooting medium) for the three microbial species; (A) Strain 1: Pantoea sp; (B) Strain 2: Curtobacterium flaccumfaciens; (C) Strain 3: Xanthomonas sp.).

FIG. 13 shows microbe growth from alginate bead, post-1 week incubation on nutrient broth medium for the three microbial species; (A) Strain 1: Pantoea sp; (B) Strain 2: Curtobacterium flaccumfaciens; (C) Strain 3: Xanthomonas sp.).

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavor to which this specification relates.

Unless specifically defined otherwise, all technical and scientific terms used herein shall be taken to have the same meaning as commonly understood by one of ordinary skill in the art.

Unless otherwise indicated the molecular biology, cell culture, laboratory, plant breeding and selection techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present); Janick, J. (2001) Plant Breeding Reviews, John Wiley & Sons, 252 p.; Jensen, N. F. ed. (1988) Plant Breeding Methodology, John Wiley & Sons, 676 p., Richard, A. J. ed. (1990) Plant Breeding Systems, Unwin Hyman, 529 p.; Walter, F. R. ed. (1987) Plant Breeding, Vol. I, Theory and Techniques, MacMillan Pub. Co.; Slavko, B. ed. (1990) Principles and Methods of Plant Breeding, Elsevier, 386 p.; and Allard, R. W. ed. (1999) Principles of Plant Breeding, John-Wiley & Sons, 240 p. The ICAC Recorder, Vol. XV no. 2: 3-14; all of which are incorporated by reference. The procedures described are believed to be well known in the art and are provided for the convenience of the reader. All other publications mentioned in this specification are also incorporated by reference in their entirety.

As used in the subject specification, the singular forms “a”, “an” and “the” include plural aspects unless the context clearly dictates otherwise. Thus, for example, reference to “a plant” includes a single plant, as well as two or more plants; reference to “a seed” includes a single seed, as well as two or more seeds; and so forth.

The present disclosure is predicated, at least in part, on the inventors' unexpected finding that isolated meristematic plant tissue derived from a cannabis plant can be encapsulated by a biocompatible polymer to produce an artificial plant seed capable of complete plant regeneration. Thus, in an aspect disclosed herein, there is provided an artificial seed comprising isolated meristematic plant tissue encapsulated by a biocompatible polymer, wherein the meristematic plant tissue is derived from a cannabis plant and sterilised prior to encapsulation.

### Artificial Seed

As used herein, the term “artificial seed” is used to denote meristematic plant tissue (e.g., somatic and/or zygotic embryos) that is encapsulated in a biocompatible substrate that allows germination of the meristematic plant tissue under suitable conditions. The artificial seed structure is intended to mimic that of the conventional seed, insofar as it contains explant material, which imitates the zygotic embryo in the conventional seed, a capsule (typically a gel agent) and, optionally, and additional materials such as nutrients, growth regulators, anti-pathogens, bio-controllers, and bio fertilizers) that emulates the endosperm in the conventional seed.

Artificial seeds allow for propagation and ongoing strain preservation, including during tissue culture. Artificial seeds can also simplify material transfer and movement of strains and genetics in a risk adverse manner. In addition, artificial seeds derived from aseptic tissue culture techniques, as herein described, allow disease, pest and/or pathogen-free plant development. Thus, the artificial seeds described herein lend themselves to propagation and production systems in which it is desirable to be insecticide and pesticide free.

### Cannabis

The terms “cannabis”, “cannabis plant” and the like are used interchangeably herein to describe a plant of the genus Cannabis, illustrative examples of which include Cannabis sativa, Cannabis indica and Cannabis ruderalis. Cannabis is an erect annual herb with a dioecious breeding system, although monoecious plants exist. Wild and cultivated forms of cannabis are morphologically variable, which has resulted in difficulty defining the taxonomic organisation of the genus. In an embodiment, the cannabis plant is C. sativa.

The terms “plant”, “cultivar”, “variety”, “strain” or “race” are used interchangeably herein to refer to a plant or a group of similar plants according to their structural features and performance (i.e., morphological and physiological characteristics).

As used herein, the term “plant part” refers to any part of the plant, illustrative examples of which include an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen. The term “plant part” also includes any material listed in the Plant Part Code Table as approved by the Australian Therapeutic Goods Administration (TGA) Business Services (TBS). In an embodiment, the part is selected from the group consisting of an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen. In a preferred embodiment, the part is a cannabis bud.

### Meristematic Plant Tissue

The term “meristematic plant tissue” or “meristem”, as used herein, means plant cells, other than a botanical seed, that is capable of giving rise to various organs of a plant and is responsible for plant growth. Meristematic plant tissue may includes apical meristems (e.g. shoot apical meristems, root apical meristems, intercalary meristems, floral meristems), primary meristems and secondary meristems. Suitable meristematic plant tissue for use in the artificial seeds described herein will be familiar to persons skilled in the art, illustrative examples of which include somatic embryos, the explants of axillary branches, nodal sections, adventitious shoots and buds apical meristems. Other illustrative examples of meristematic plant tissue are described by the review of Rihan et al. (Agronomy, 2017, 7:71; “Artificial Seeds (Principle, Aspects and Applications)”), the contents of which are incorporated herein by reference.

Meristematic plant tissue can therefore be any plant tissue, or group of plant cells, which is capable of developing into a complete plant or part of a complete plant when subjected to suitable conditions. This term embraces any type of plant tissue, illustrative examples of which include callus tissue, protoplasts, plant organs, zygotic embryos, somatic tissue, somatic embryos, zygotic tissue, germs, adventitions, buds, shoots, shoot primordia, protocorm-like bodies, green spots, the germ line, and young seedlings. Meristematic plant tissue may comprise undifferentiated plant cells that divide to yield other meristematic cells and/or differentiated cells that elongate and further specialize to form structural tissues and organs of the plant. Meristematic plant tissue may be located, for example, at the extreme tips of growing shoots or roots, in buds, and in the cambium layer of woody plants. In an embodiment disclosed herein, the meristematic plant tissue is selected from the group consisting of plant cells, callus tissue, a protoplast, a plant organ, a zygotic embryo and a somatic embryo. Plant embryonic tissue can be found (in the form of a “zygotic” embryo) inside a botanic seed produced by sexual reproduction. Also, plant “somatic” embryos can be produced by culturing totipotent plant cells such as meristematic plant tissue under laboratory conditions in which the cells comprising the tissue are separated from one another and urged to develop into minute complete embryos.

Suitable plant organs for use in generating the artificial seeds, as described herein, will also be familiar to persons skilled in the art, illustrative examples of which include adventitious shoots, micronodules, axillary buds, apical buds and scions. In an embodiment disclosed herein, the plant organ comprises an adventitious shoot, a micronodule, an axillary bud, an apical bud and/or a scion. In a preferred embodiment, the plant organ comprises an axillary bud.

In an embodiment, meristematic plant tissue is plant tissue that can be individually handled and encapsulated by the biocompatible polymer as herein described, and which will develop into a germinant and ultimately a cannabis plant or plantlet under favourable conditions.

### Biocompatible Polymer

The term “biocompatible polymer”, as used herein, typically means a synthetic or natural polymer that is substantially inert, insofar as it will have substantially no or minimal adverse effect on the health of the plant cells to which it may come in contact, or on the development of a plant from the meristematic plant tissue encapsulated therein; that is, the biocompatible polymer is suitably neither cytotoxic nor substantially phytotoxic. As used herein, a “substantially non-phytotoxic” substance is a substance that does not interfere substantially with normal plant development, such as by killing a substantial number of plant cells, substantially altering cellular differentiation or maturation, causing mutations, disrupting a substantial number of cell membranes or substantially disrupting cellular metabolism, or substantially disrupting other process. Suitable biocompatible polymers will be familiar to persons skilled in the art, illustrative examples of which include alginates, guar gums, agar, agarose, gelatin, starch, polyacrylamide, and other gels. In an embodiment, the biocompatible polymer is selected from the group consisting of gelatin, casein, arabinoxylan, soluble starch, chitin, pectin, alginate, methyl cellulose, hydroxyethyl cellulose, methylhydroxypropyl cellulose, methylhydroxyethyl cellulose, hydroxylpropyl cellulose, sucrose and mixtures thereof. In an embodiment, the biocompatible polymer comprises alginate. Suitable forms of alginate will be familiar to persons skilled in the art, illustrative examples of which include sodium alginate and calcium alginate. Thus, in an embodiment, the alginate is sodium alginate or calcium alginate.

Suitable methods for encapsulating meristematic plant tissue in a biocompatible polymer will be familiar to persons skilled in the art, illustrative examples of which are described by, or referred to in, Rihan et al. (Agronomy, 2017, 7:71; “Artificial Seeds (Principle, Aspects and Applications)”). For example, as described elsewhere herein, the meristematic plant tissue can be coated in a solution of the biocompatible polymer and subsequently exposed to a complexing agent that will facilitate the solidification of the biocompatible polymer to form a biocompatible polymer gel encapsulating the plant tissue. A sodium alginate solution, for instance, will form a gel when a complexing agent is added. Calcium chloride (CaCl₂) is generally used as a complexing agent (or gelling agents) for sodium alginate, however, lanthanum chloride, ferric chloride, cobaltous chloride, calcium nitrate, calcium hydroxide, superphosphate fertilizer, and many pesticides such as benefin, alachlor and chlorpropham are also generally suitable, as are other multivalent cation compounds.

Persons skilled in the art will understand that a variety of biocompatible polymer compositions may be used to produce the artificial seeds described herein, whether compositions may vary in the concentration of the biocompatible polymer solution in which the meristematic plant tissue is to be coated. The concentration of the biocompatible polymer will suitably be chosen with a view to optimizing ease of handling, gelling time, strength of gel and coating thickness around the encapsulated material, having regard to factors such as the intended use of the artificial seed and the proposed length and/or temperature of storage. For instance, if the biocompatible polymer is too dilute, the encapsulated material can settle during gel formation and produce an uneven encapsulation. It would be within the capability of persons skilled in the art to modify the concentration of the biocompatible polymer in solution to achieve the desired gelling time, strength and coating thickness around the meristematic plant tissue.

The concentration of biocompatible polymer in solution required to prepare a satisfactory gel for encapsulation of the meristematic plant tissue, as described herein, will likely vary depending upon the particular biocompatible polymer. In general, gels cured by complexing require less gel solute to form a satisfactory gel than “reversible” gels.

In an embodiment, the sodium alginate is prepared in a concentration from about 1 to about 10% weight to volume (w/v) in water, preferably from about 2 to about 10% w/v, or more preferably from about 3 to about 5% w/v. As used herein, “% w/v” is equivalent to grams of solute per 100 mL of solvent.

The calcium chloride (or other suitable complexing agent) may be made up in solution at a concentration that is determined to be suitable for solidifying the biocompatible polymer solution about the meristematic plant tissue. Suitable concentrations of complexing agent will be familiar to persons skilled in the art and likely to depend on the type of biocompatible polymer used to encapsulating the meristematic plant tissue. In an embodiment, the complexing agent is calcium chloride. In an embodiment, the concentration of the calcium chloride is from about 1 to about 1,000 millimolar, preferably from about 20 to about 500 millimolar, or more preferably from about 50 to about 300 millimolar. Other complexing agents will have different preferred concentration ranges, as will be known to persons skilled in the art.

The time for gel formation and temperature of the gelling solutions (i.e., complexing agents) are interrelated parameters, for the selected concentrations of biocompatible polymer and complexing agent. In an embodiment, the temperature chosen can be in the range of about 1° to about 50° C., preferably from about 10° to about 40° C. or more preferably from about 20° to about 40° C. Within the range of acceptable temperatures, a particular value may be chosen to give the shortest possible gelling time consistent with complete gel formation. Typically, the gel will form immediately, but the complexation takes much longer. For a solution of sodium alginate at a concentration of about 3.2 grams per 100 milliliters H₂O, a calcium chloride solution concentration of about 50 millimolar, and a reaction temperature of about 25° C., adequate gelling is obtained in about 5 to about 120 minutes, more often in about 10 to about 90 minutes and is usually sufficiently complete in about 30 to about 60 minutes. Alternatively, if using about 50 millimolar calcium chloride, gelation time is likely to decrease to about 2-5 minutes.

In an embodiment, the thickness of the encapsulating biocompatible polymer is from about 0.1 to about 5 mm, more preferably from about 0.25 to about 1.5 mm in thickness.

As described elsewhere herein, the biocompatible polymer characteristics described above can be modifiable for each gel, and are likely to be determined generally by the concentration parameters and chemical properties of the gel.

The biocompatible polymer may suitably comprise additives to assist in plant development, such as plant nutrients, pesticides, and hormones. In an embodiment, wherein the biocompatible polymer comprises a polysaccharide. Suitable polysaccharides for use in an artificial seed, as herein described, will be familiar to persons skilled in the art, illustrative example of which include sucrose, lactose and maltose. In an embodiment, the polysaccharide is sucrose.

The artificial seeds described herein may comprise a variety of other additives and/or adjuvants, the nature of which is generally determined by the intended use, the type of meristematic plant tissue, and so on. Suitable additives and/or adjuvants will be familiar to persons skilled in the art, illustrative examples of which include fertilizers, fungicides, bactericides, trace elements, and nutrients. These additives and/or adjuvants can be incorporated into the biocompatible polymer prior to gelling and/or incorporated into the biocompatible polymer gel subsequent to solidification. In other embodiments, the additives and/or adjuvants can be incorporated in between two layers of biocompatible polymer gels encapsulating the meristematic plant tissue. Typically, the additives and/or adjuvants will be chosen and incorporated into the biocompatible polymer prior to encapsulation concentrations that will not substantially interfere with gelling. Suitably, a cured biocompatible polymer gel will have sufficient strength to maintain the integrity of the artificial seed capsule without the capsule being so durable that a germinating embryo cannot penetrate it.

As used herein, the term “gel” means a substance that is prepared as a colloidal solution and that will, or can be caused to, form a semisolid material. Such conversion of a liquid biocompatible polymer solution into a semisolid material is often referred to as “curing”, “setting” or “solidifying” the gel.

The biocompatible polymer gels, as described herein, are typically prepared by dissolving a gel solute, usually in fine particulate form, in water to form a gel solution. Depending upon the particular biocompatible polymer (gel) solute, heating is usually necessary, sometimes to boiling, before the gel solute will dissolve. Subsequent cooling will cause many gel solutions to reversibly “set” or “cure” (become gelled). Illustrative examples include gelatin, agar, and agarose, which are often referred to as “reversible” because reheating cured gel will re-form the gel solution. As noted elsewhere herein, solutions of other biocompatible polymer solutes require a “complexing” agent which serves to chemically cure the gel by crosslinking gel solute molecules. For example, sodium alginate is cured by adding calcium nitrate calcium chloride, or salts of other divalent ions such as, but not limited to, calcium, barium, lead, copper, strontium, cadmium, zinc, nickel, cobalt, magnesium, and iron to the gel solution.

It is generally desirable to provide the meristematic plant tissue with suitable plant nutrients and other beneficial substances such as vitamins and a source of carbon and energy (herein collectively termed generally “nutrients”) while the meristematic plant tissue is encapsulated in the biocompatible polymer gel. Typical ways of providing nutrients in this manner will be familiar to persons skilled in the art, an illustrative example of which is to dissolve the biocompatible polymer solute in a solution of the nutrients or to add a volume of concentrated nutrient solution to the biocompatible polymer solution before curing. In this way, when the gel sets (“cures”), any areas of the meristematic plant tissue that is in contact with the biocompatible polymer gel are also in direct contact with nutrient solutes, where the nutrient solutes are suitably present in substantially uniform concentrations throughout the biocompatible polymer gel. Another illustrative example by which to provide nutrients to the meristematic plant tissue is to place a biocompatible polymer gel capsule containing the meristematic plant tissue, but lacking nutrients, in contact with a second mass of the same or a different type of biocompatible polymer gel which contains nutrients. As a result of a nutrient concentration gradient between the two biocompatible polymer gels, nutrients will migrate from the nutrient-containing gel to the gel encapsulating the meristematic plant material.

Another possible way to provide nutrients is to place a biocompatible polymer gel encapsulating the meristematic plant tissue, but lacking nutrients, in contact with a second substrate comprising microencapsulated nutrients or nutrients associated with any substantially non-phytotoxic substrate that will allow the nutrients dissolved therein to be transferred via water to the plant tissue-encapsulating biocompatible polymer gel. Representative materials include, but are not limited to, water, a biocompatible polymer gel similar to the biocompatible polymer gel encapsulating the meristematic plant tissue, vermiculite, perlite, or any suitable polymeric material that is non-toxic and is capable of releasing the nutrients readily over a period of time.

It may be desirable, in some instances, to encapsulate the meristematic plant tissue in more than one layer of biocompatible polymer. This may be desirable, for example, where a greater thickness is required, having regard to, for example, intended use, storage land and storage temperatures. In an embodiment, the meristematic plant tissue is encapsulated by at least two layers of biocompatible polymers. By “at least two layers of biocompatible polymers” is meant at least 2, preferably at least 3, or more preferably at least 4 layers of biocompatible polymers. It is to be understood that each layer of biocompatible polymer encapsulating the meristematic plant tissue may be formed of the same type of biocompatible polymer or, alternatively, each layer may be formed of different type of biocompatible polymer. In an embodiment, each of the at least two layers comprises the same biocompatible polymer.

### Endophytes

As is described elsewhere herein, the inventors' have also surprisingly found that co-encapsulating the isolated meristematic plant tissue with an endophyte enables the endophytes to colonise the plants during the plant regeneration process. Thus, in an embodiment disclosed herein, the biocompatible polymer comprises an endophyte. In an embodiment, where the artificial seed comprises at least two layers of biocompatible polymers, at least one of the layers of biocompatible polymers comprises an endophyte. In an embodiment, the meristematic plant tissue is encapsulated by an inner layer of a first biocompatible polymer and an outer layer of a second biocompatible polymer, and wherein the inner layer comprises an endophyte.

The term “endophyte”, as used herein, means a microbe (e.g., a fungus or bacterium) that lives between living plant cells, and will typically exist symbiotically and/or asymptomatically with the plant cells.

Suitable endophytes will be familiar to persons skilled in the art, illustrative examples of which include fungal and bacterial species. In an embodiment, the endophyte is one or more fungal species. In an embodiment, the endophyte is one or more bacterial species. In an embodiment, the endophyte is one or more fungal species and one or more bacterial species. Fungal and bacterial species suitable for users endophytes for plant development, in particular cannabis plant development, will be familiar to persons skilled in the art, illustrative examples of which include Achromobacter, Acidovorax, Acinetobacter, Actinoplanes, Advenella, Aeromicrobium, Agreia, Agrobacterium, Alloprevotella, Anabaena, Anaerococcus, Aquabacterium, Arcicella, Arthrobacter, Averyella, Azospirillum, Bacillus, Bdellovibrio, Beggiatoa, Brachybacterium, Brevundimonas, Bryobacter, Burkholderia, Buttiauxella, Caenimonas, Campylobacter, Chloracidobacterium, Candidatus Microthrix, Castellaniella, Cellulomonas, Cellvibrio, Chryseobacterium, Chthoniobacter, Citrobacter, Clavibacter, Clostridium, Comamonas, Corynebacterium, Coxiella, Cronobacter, Cryocola, Cupriavidus, Curtobacterium, Cytophaga, Dechloromonas, Deinococcus, Delftia, Devosia, Diaminobutyricimonas, Dokdonella, Dongia, Duganella, Enterobacter, Enterococcus, Erwinia, Escherichia, Ferrovibrio, Ferruginibacter, Flavobacterium, Flexibacter, Fluviicola, Frigoribacterium, Fusobacterium, Gaiella, Galbitalea, Gemmata, Gemmatimonas, Geobacter, Giesbergeria, Haliangium, Herbaspirillum, Hirschia, Hydrogenophaga, Inhella, Janthinobacterium, Kineococcus, Klebsiella, Kluyvera, Kosakonia, Kytococcus, Lacibacter, Lactobacillus, Lactococcus, Lautropia, Legionella, Leifsonia, Lelliottia, Leptolyngbya, Leptospira, Leptothrix, Limnohabitans, Luteibacter, Luteimonas, Luteolibacter, Lysinimonas, Lysobacter, Marmoricola, Massilia, Methylobacterium, Methylophilus, Methylotenera, Methyloversatilis, Microbacterium, Micrococcus, Mycobacterium, Neisseria, Nevskia, Niastella, Nitrosomonas, Niveispirillum, Nocardioides, Nostoc, Novosphingobium, Ochrobactrum, Oligoflexus, Opitutus, Oscillatoria, Paenarthrobacter, Paenibacillus, Paludibaculum, Pantoea, Pediococcus, Pedobacter, Peredibacter, Pigmentiphaga, Pirellula, Planctomyces, Prevotella, Propionibacterium, Prosthecobacter, Providencia, Pseudarthrobacter, Pseudohongiella, Pseudomonas, Pseudorhodoferax, Pseudoxanthomonas, Quadrisphaera, Ralstonia, Ramlibacter, Rathayibacter, Reyranella, Rheinheimera, Rhizobium, Rhizomicrobium, Rhodanobacter, Rhodopirellula, Roseiflexus, Roseomonas, Rothia, Rummeliibacillus, Runella, Saccharibacillus, Salinibacterium, Salmonella, Sanguibacter, Segniliparus, Serratia, Shigella, Sodalis, Solirubrobacter, Sphingobacterium, Sphingomonas, Sphingopyxis, Spirosoma, Stenotrophomonas, Stenotrophomonas, Steroidobacter, Streptococcus, Streptomyces, Streptophyta, Tatumella, Thermomonas, Trabulsiella, Trichormus, Tsukamurella, uncultured, Variovorax, Veillonella, Verticia, Wautersiella, Weissella, Xanthomonas, Xylella, Xylophilus, Yonghaparkia, Acremonium, Alternaria, Amorphotheca, Anthracocystis, Apiotrichum, Aplosporella, Apodus, Aspergillus, Aureobasidium, Beauveria, Bipolaris, Candida, Capnodiales, Cercospora, Chaetomium, Chrysosporium, Cladosporium, Clonostachys, Cochliobolus, Coniochaeta, Coniothyrium, Coprinopsis, Corynascella, Cryptococcus, Curvularia, Daldinia, Emericellopsis, Ephelis, Epichloe, Epicoccum, Eurotiales, Exserohilum, Fusarium, Geomyces., Gibberella, Helotiales, Kazachstania, Khuskia, Lecythophora, Leohumicola, Leptosphaerulina, Magnaporthe, Microdiplodia, Microdochium, Microsphaeropsis, Mucor, Muscador, Nodulisporium, Oidiodendron, Ophiosphaerella, Papiliotrema, Paraconiothyrium, Penicillium, Phaeosphaeria, Phaeosphaeriopsis, Phialemonium, Phoma, Pithomyces, Pleosporales., Pseudogymnoascus, Pseudozyma, Pyrenochaetopsis, Ramichloridium, Rhizomucor, Sarocladium, Scopulariopsis, Simplicillium, Sordariales, Sporisorium, Thielavia, Trichosporon, Ustilaginales, Ustilago, Waitea and Xylariales.

In an embodiment, the one or more bacterial species is selected from the group consisting of Achromobacter, Acidovorax, Acinetobacter, Actinoplanes, Advenella, Aeromicrobium, Agreia, Agrobacterium, Alloprevotella, Anabaena, Anaerococcus, Aquabacterium, Arcicella, Arthrobacter, Averyella, Azospirillum, Bacillus, Bdellovibrio, Beggiatoa, Brachybacterium, Brevundimonas, Bryobacter, Burkholderia, Buttiauxella, Caenimonas, Campylobacter, Chloracidobacterium, Candidatus Microthrix, Castellaniella, Cellulomonas, Cellvibrio, Chryseobacterium, Chthoniobacter, Citrobacter, Clavibacter, Clostridium, Comamonas, Corynebacterium, Coxiella, Cronobacter, Cryocola, Cupriavidus, Curtobacterium, Cytophaga, Dechloromonas, Deinococcus, Delftia, Devosia, Diaminobutyricimonas, Dokdonella, Dongia, Duganella, Enterobacter, Enterococcus, Erwinia, Escherichia, Ferrovibrio, Ferruginibacter, Flavobacterium, Flexibacter, Fluviicola, Frigoribacterium, Fusobacterium, Gaiella, Galbitalea, Gemmata, Gemmatimonas, Geobacter, Giesbergeria, Haliangium, Herbaspirillum, Hirschia, Hydrogenophaga, Inhella, Janthinobacterium, Kineococcus, Klebsiella, Kluyvera, Kosakonia, Kytococcus, Lacibacter, Lactobacillus, Lactococcus, Lautropia, Legionella, Leifsonia, Lelliottia, Leptolyngbya, Leptospira, Leptothrix, Limnohabitans, Luteibacter, Luteimonas, Luteolibacter, Lysinimonas, Lysobacter, Marmoricola, Massilia, Methylobacterium, Methylophilus, Methylotenera, Methyloversatilis, Microbacterium, Micrococcus, Mycobacterium, Neisseria, Nevskia, Niastella, Nitrosomonas, Niveispirillum, Nocardioides, Nostoc, Novosphingobium, Ochrobactrum, Oligoflexus, Opitutus, Oscillatoria, Paenarthrobacter, Paenibacillus, Paludibaculum, Pantoea, Pediococcus, Pedobacter, Peredibacter, Pigmentiphaga, Pirellula, Planctomyces, Prevotella, Propionibacterium, Prosthecobacter, Providencia, Pseudarthrobacter, Pseudohongiella, Pseudomonas, Pseudorhodoferax, Pseudoxanthomonas, Quadrisphaera, Ralstonia, Ramlibacter, Rathayibacter, Reyranella, Rheinheimera, Rhizobium, Rhizomicrobium, Rhodanobacter, Rhodopirellula, Roseiflexus, Roseomonas, Rothia, Rummeliibacillus, Runella, Saccharibacillus, Salinibacterium, Salmonella, Sanguibacter, Segniliparus, Serratia, Shigella, Sodalis, Solirubrobacter, Sphingobacterium, Sphingomonas, Sphingopyxis, Spirosoma, Stenotrophomonas, Stenotrophomonas, Steroidobacter, Streptococcus, Streptomyces, Streptophyta, Tatumella, Thermomonas, Trabulsiella, Trichormus, Tsukamurella, uncultured, Variovorax, Veillonella, Verticia, Wautersiella, Weissella, Xanthomonas, Xylella, Xylophilus and Yonghaparkia.

In an embodiment, the one or more fungal species is selected from the group consisting of Acremonium, Alternaria, Amorphotheca, Anthracocystis, Apiotrichum, Aplosporella, Apodus, Aspergillus, Aureobasidium, Beauveria, Bipolaris, Candida, Capnodiales, Cercospora, Chaetomium, Chrysosporium, Cladosporium, Clonostachys, Cochliobolus, Coniochaeta, Coniothyrium, Coprinopsis, Corynascella, Cryptococcus, Curvularia, Daldinia, Emericellopsis, Ephelis, Epichloe, Epicoccum, Eurotiales, Exserohilum, Fusarium, Geomyces., Gibberella, Helotiales, Kazachstania, Khuskia, Lecythophora, Leohumicola, Leptosphaerulina, Magnaporthe, Microdiplodia, Microdochium, Microsphaeropsis, Mucor, Muscador, Nodulisporium, Oidiodendron, Ophiosphaerella, Papiliotrema, Paraconiothyrium, Penicillium, Phaeosphaeria, Phaeosphaeriopsis, Phialemonium, Phoma, Pithomyces, Pleosporales., Pseudogymnoascus, Pseudozyma, Pyrenochaetopsis, Ramichloridium, Rhizomucor, Sarocladium, Scopulariopsis, Simplicillium, Sordariales, Sporisorium, Thielavia, Trichosporon, Ustilaginales, Ustilago, Waitea and Xylariales.

### Method of Producing an Artificial Plant Seed

In another aspect disclosed herein, there is provided a method of producing an artificial plant seed of meristematic cannabis plant tissue encapsulated by a biocompatible polymer, the method comprising:
(a) isolating meristematic plant tissue from a cannabis plant;
(b) sterilizing the isolated meristematic plant tissue of (a);
(c) coating the sterilized meristematic plant tissue of (b) in a biocompatible polymer solution; and
(d) exposing the polymer-coated meristematic plant tissue of (c) to a complexing agent to solidify the biocompatible polymer, thereby encapsulating the isolated meristematic cannabis plant tissue with the biocompatible polymer.

Suitable methods of sterilizing plant tissue, including meristematic plant tissue, will be familiar to persons skilled in the art, illustrative examples of which are described elsewhere herein (e.g., immersing the plant material in an alcohol solution, such as 80% ethanol/water and/or sodium hypochlorite).

In another aspect disclosed herein, there is provided a method of producing an artificial plant seed of meristematic cannabis plant tissue encapsulated by a biocompatible polymer, the method comprising:
(a) isolating meristematic plant tissue from a cannabis plant;
(b) sterilizing the isolated meristematic plant tissue of (a);
(c) coating the sterilized meristematic plant tissue of (b) in a biocompatible polymer solution; and
(d) exposing the polymer-coated meristematic plant tissue of (c) to a complexing agent to solidify the biocompatible polymer, thereby encapsulating the isolated meristematic cannabis plant tissue with the biocompatible polymer.

In an embodiment, the meristematic plant tissue is selected from the group consisting of plant cells, callus tissue, protoplasts, a plant organ, a zygotic embryo and a somatic embryo.

In an embodiment, the plant organ comprises an adventitious shoot, a micronodule, an axillary bud, an apical bud and/or a scion.

In an embodiment, the plant organ comprises an axillary bud.

In an embodiment, the method comprises:
(c) repeating steps (c) and (d), as herein described, to further encapsulate the meristematic plant tissue in an outer layer of a biocompatible polymer, thereby encapsulating the isolated meristematic plant tissue in an inner layer of a first biocompatible polymer and in an outer layer of a second biocompatible polymer.

In an embodiment, the biocompatible polymer comprises an endophyte.

In an embodiment, the inner layer of the biocompatible polymer comprises an endophyte.

In an embodiment, the endophyte is one or more fungal species.

In an embodiment, the endophyte is one or more bacterial species.

In an embodiment, the endophyte is one or more fungal species and one or more bacterial species.

In an embodiment, the one or more bacterial species is selected from the group consisting of Achromobacter, Acidovorax, Acinetobacter, Actinoplanes, Advenella, Aeromicrobium, Agreia, Agrobacterium, Alloprevotella, Anabaena, Anaerococcus, Aquabacterium, Arcicella, Arthrobacter, Averyella, Azospirillum, Bacillus, Bdellovibrio, Beggiatoa, Brachybacterium, Brevundimonas, Bryobacter, Burkholderia, Buttiauxella, Caenimonas, Campylobacter, Chloracidobacterium, Candidatus Microthrix, Castellaniella, Cellulomonas, Cellvibrio, Chryseobacterium, Chthoniobacter, Citrobacter, Clavibacter, Clostridium, Comamonas, Corynebacterium, Coxiella, Cronobacter, Cryocola, Cupriavidus, Curtobacterium, Cytophaga, Dechloromonas, Deinococcus, Delftia, Devosia, Diaminobutyricimonas, Dokdonella, Dongia, Duganella, Enterobacter, Enterococcus, Erwinia, Escherichia, Ferrovibrio, Ferruginibacter, Flavobacterium, Flexibacter, Fluviicola, Frigoribacterium, Fusobacterium, Gaiella, Galbitalea, Gemmata, Gemmatimonas, Geobacter, Giesbergeria, Haliangium, Herbaspirillum, Hirschia, Hydrogenophaga, Inhella, Janthinobacterium, Kineococcus, Klebsiella, Kluyvera, Kosakonia, Kytococcus, Lacibacter, Lactobacillus, Lactococcus, Lautropia, Legionella, Leifsonia, Lelliottia, Leptolyngbya, Leptospira, Leptothrix, Limnohabitans, Luteibacter, Luteimonas, Luteolibacter, Lysinimonas, Lysobacter, Marmoricola, Massilia, Methylobacterium, Methylophilus, Methylotenera, Methyloversatilis, Microbacterium, Micrococcus, Mycobacterium, Neisseria, Nevskia, Niastella, Nitrosomonas, Niveispirillum, Nocardioides, Nostoc, Novosphingobium, Ochrobactrum, Oligoflexus, Opitutus, Oscillatoria, Paenarthrobacter, Paenibacillus, Paludibaculum, Pantoea, Pediococcus, Pedobacter, Peredibacter, Pigmentiphaga, Pirellula, Planctomyces, Prevotella, Propionibacterium, Prosthecobacter, Providencia, Pseudarthrobacter, Pseudohongiella, Pseudomonas, Pseudorhodoferax, Pseudoxanthomonas, Quadrisphaera, Ralstonia, Ramlibacter, Rathayibacter, Reyranella, Rheinheimera, Rhizobium, Rhizomicrobium, Rhodanobacter, Rhodopirellula, Roseiflexus, Roseomonas, Rothia, Rummeliibacillus, Runella, Saccharibacillus, Salinibacterium, Salmonella, Sanguibacter, Segniliparus, Serratia, Shigella, Sodalis, Solirubrobacter, Sphingobacterium, Sphingomonas, Sphingopyxis, Spirosoma, Stenotrophomonas, Stenotrophomonas, Steroidobacter, Streptococcus, Streptomyces, Streptophyta, Tatumella, Thermomonas, Trabulsiella, Trichormus, Tsukamurella, uncultured, Variovorax, Veillonella, Verticia, Wautersiella, Weissella, Xanthomonas, Xylella, Xylophilus and Yonghaparkia.

In an embodiment, the one or more fungal species is selected from the group consisting of Acremonium, Alternaria, Amorphotheca, Anthracocystis, Apiotrichum, Aplosporella, Apodus, Aspergillus, Aureobasidium, Beauveria, Bipolaris, Candida, Capnodiales, Cercospora, Chaetomium, Chrysosporium, Cladosporium, Clonostachys, Cochliobolus, Coniochaeta, Coniothyrium, Coprinopsis, Corynascella, Cryptococcus, Curvularia, Daldinia, Emericellopsis, Ephelis, Epichloe, Epicoccum, Eurotiales, Exserohilum, Fusarium, Geomyces., Gibberella, Helotiales, Kazachstania, Khuskia, Lecythophora, Leohumicola, Leptosphaerulina, Magnaporthe, Microdiplodia, Microdochium, Microsphaeropsis, Mucor, Muscador, Nodulisporium, Oidiodendron, Ophiosphaerella, Papiliotrema, Paraconiothyrium, Penicillium, Phaeosphaeria, Phaeosphaeriopsis, Phialemonium, Phoma, Pithomyces, Pleosporales., Pseudogymnoascus, Pseudozyma, Pyrenochaetopsis, Ramichloridium, Rhizomucor, Sarocladium, Scopulariopsis, Simplicillium, Sordariales, Sporisorium, Thielavia, Trichosporon, Ustilaginales, Ustilago, Waitea and Xylariales.

In another aspect disclosed herein, there is provided an artificial seed produced by the methods described herein.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

The various embodiments enabled herein are further described by the following non-limiting examples.

### EXAMPLES

### Example 1

### Preparation of Artificial Seeds of Cannabis Sativa

In this study, axillary bud was used as the meristematic plant tissue. Cannabis explants can be used direct post-sterilisation or post-storage, for up to 3 hours stored at 4° C. before proceeding with axillary bud encapsulation.

### Materials and Methods:

Ethanol [80%].
Milli-Q water [Sterile].
Tween® 20 [Sigma #P9416].
Domestos® [Available Chlorine 4.75% m/v].
Chemicals required for media preparation:
   Sucrose grade II® [Sigma-Aldrich #S5391].
   Indol-3-butyric acid® [Sigma-Aldrich #15386].
   Agar® Type E, plant cell culture tested [Sigma-Aldrich #A4675].
   Alginic acid sodium salt from brown algae [Sigma-Aldrich #A2033].
   Murashige & Skoog medium including vitamins® [Duchefa-Biochemie #M0222].
Laboratory equipment required:
   Orbital shaker.
   Sterilizer, dry bead.
   Pipette filler electronic.
   Air laminar flow cabinet.
   Digital camera.
   CER [Controlled Environment/Atmosphere Room].
Laboratory instruments and consumables required:
   Scalpel handles no. 7 [Sterile].
   Scalpel blades no. 11 [Sterile].
   Curved forceps 200 mm [Sterile].
   Petri dishes 20×100 mm [Sterile].
   Cylinder 100 ml and 1 L Glass beaker.
   Tub 946 ml SteriCon™-8, culture vessel [Sterile].
   Corning® 6 well non-treated plate [Ref #351146] [Sterile].

### Preparation and Sterilization of Cannabis Explants

### A. Sterilization Stage 1: Plant Tissue Selection and Initial Cleaning:

a) Select and excise the appropriate tissue as a cutting from a mature healthy mother plant. The tissue should be free from necrosis and any other signs of infection or general poor health. Excise the cutting from between the axillary buds from the donor plant stem. Trim the axillary buds to minimise the explant size to 3 mm by eliminating any growing leaves. Rinse the cannabis explants several times under running tap water.
b) Surface sterilize the cannabis explants by stirring the plant tissue in 80% Ethanol (v/v) for 1 minute.
c) Decant the Ethanol and rinse the cannabis explants with tap water at least three times.
d) Surface sterilize cannabis explants by immersing in 15% Domestos® [4.75% available Chlorine m/v] that has had the addition of 2-3 drops of Tween 20, for 15 min with shaking at 150 rpm/min.

### B. Sterilization Stage 2: Aseptic Treatment of Plant Tissue:

a) Decant the Domestos and rinse the explants several times with sterile Milli-Q water.
b) Continue rinsing the cannabis explants by sterile Milli-Q water to remove all traces of the sterilizing agent [indicated by no white foam remaining around the explants].
c) Retain the sterilized cannabis explants in sterile Milli-Q water for 2-5 minutes. Trim buds to c. 3 mm size using aseptic techniques prior to transfer to gel encapsulation.
d) Transfer the sterilized cannabis explants to 100 ml flask with 50 ml of gel matrix.

### In Vitro Culture of Encapsulated Axillary Buds

a) Mix the post-sterilized axillary buds with the gel matrix [Table 1] in a 100 ml conical flask and follow the following steps.
b) Mix the trimmed buds [3 mm] with 50 ml of gel matrix in a flask covered by aluminium foil.
c) Drop the encapsulated explants [mixed with Sodium Alginate gel matrix] into 50-100 mM CaCl₂.2H₂O. Stir the explants through the use of an orbital shaker at 80 rpm/30 min to boost the beads formation from Sodium Alginate.
d) Discard the CaCl₂.2H₂O and transfer the encapsulated explants to shoot proliferation media.
e) Incubate the encapsulated explants to shoot proliferation media [Table 2] for 1 to 2 weeks in controlled environment rooms [CER] at 25±1° C./16 hr light.

TABLE 1
Encapsulation gelling matrix medium
Encapsulation Gelling Matrix Composilion

| Compositton | g/100 ml |
|---|---|
| Sodium Alginate (5%) | 5 gm |
| Sucrose grade II (7.5%) | 7.5 gm |

TABLE 2
Shoot proliferation medium
Shoot Proliferation Media Composition

| Composition | g/L | |
|---|---|---|
| Murashige and Skoog Basal Medium with MS | 4.4 | gm |
| Vitamins | | |
| Sucrose grade II (3%) | 30 | gm |

pH Conditions

| pH | Buffer/s | | |
|---|---|---|---|
| 5.7 | 1M NaOH/HCl | | |
| Gelling agent | Final concentrationg | g/L | |
| Plant Agar | 0.8% | 8 | gm |

Method of Sterilisation
Autoclave 121 PSI/16 min
Hormones

| Component | Final conc. | Stock | V/L | |
|---|---|---|---|---|
| IBA | 0.05 mg/l | 1.0mg/ml | 50 | μl |

Container Details for Dispensing
20x 100 ml SteriCon ™ sterilised tub
Media Pour Details
30-35 ml/ plate
Storage Requirements
Room temperature or 4° C. cold room storage

### Example 2

### Storage of Artificial Seeds of Cannabis Sativa

Following the generation and formation of the alginate bead surrounding the axillary bud that has created the artificial seed, transfer to an appropriate sterile plastic storage vessel and transfer to 4° C. Storage at this temperature can be maintained for three (3) weeks. After three (3) weeks the artificial seeds are removed from incubation at 4° C. and are entered into the plant regeneration protocol without alteration. A 30% increase in mortality rate is observed for the addition of this protocol compared to the standard methodology.

### Example 3

### Regeneration of Artificial Seeds of Cannabis Sativa

### Planting Out Instruments and Consumables Required

1. Small plastic scoop.
2. Coconut peat 25-30 L.
3. Plastic containers 1 L.
4. Warm tap water 55° C.
5. Coir 3.5-inch [Jiffy pots].
6. Non-sterile gloves [Large].
7. 30 cm long curved forceps.
8. Long rounds tub with 2 vents.
9. Compact towel [90 towels/pack].
10. Vermiculite gold [grade #2] 10-15 L.
11. Garden dibber tool [plastic or wood].
12. Propagator lid with vents fit for seedling tray.
13. Drip Tray [35 cm (L)×29 cm (W)×5.5 cm (D)].
14. Seedling Tray [34 cm (L)×29 cm (W)×5.5 cm (D)].
15. Plastic pot 4-inch with 4 holes on side-wall for drainage.
16. THC [Total Horticultural Concentrate; a complete advanced plant food].

### Subculture the Regenerated Axillary Bud's to Rooting Media

Subculture the regenerated axillary buds to fresh shooting/rooting solid medium [Table 3] after 7 days and thereafter every 1-2 weeks. Once the plantlets have developed a well-developed root system they are removed from the culture vessel and medium. All tissue culture medium is then removed from the roots using long-curved forceps and a scalpel with the addition of warm water to assist, to leave clean exposed roots.

Transfer the plantlet to a 3-inch Jiffy pot that contains a mix of coconut peat and vermiculite gold [grade#2] [1:1] or [2:1] for further growth. Place the 3-inch Jiffy pot inside the 4-inch round plastic pot. Place 5 pots per each seedling tray and cover the seedling tray with a transparent lid, or with a round tub [ø110 mm×140 mm (H)] per pot, keep the ventilation lid holes fasten up to 4 days then gradually open the ventilation till to remove the lid's post around 7 days.

Maintain a high humidity within the seedling tray and water the plantlets daily without over watering. Transfer the regenerated cannabis plants post 3-4 weeks from the 3-inch Jiffy pot to individual 10-inch plastic pot [1 plant/pot].

TABLE 3
Cannabis shooting/rooting medium
Cannabis Shooting/Rooting Media Composition

| Composition | g IL | |
|---|---|---|
| Murashige and Skoog Basal Medium with MS | 2.2 | gm |
| Vitamins [Half strength] | | |
| Sucrose grade II (1%) | 10 | gm |

pH Conditions

| pH | Buffer/s | | |
|---|---|---|---|
| 5.7 | 1M NaOH/HCl | | |
| Gelling agent | Final concentration | g/L | |
| Plant Agar | 1.0% | 10 | gm |

Method of Sterilisation
Autoclave 121 PSI/16 min
Hormones

| Component | Final conc. | Stock | V/L | |
|---|---|---|---|---|
| IBA | 1.0 mg/l | 1.0 mg/ml | 1000 | μl |

Container Details for Dispensing
946 ml SteriConlm ™sterilised tub
Media Pour Details
100-120 ml/Tub
Storage Requirements
Room temperature or 4° C. cold room storage

### Example 4

### Encapsulation and Grow-Out of Microbes in Synthetic Seeds

A range of microbes (endophytes) were selected for inoculation into sodium alginate beads to exemplify the process. The microbes selected were of the varieties Pantoea sp.; Xanthomonas sp.; and Curtobacterium flaccumfaciens

The bacterial strains were cultured on Nutrient Broth medium (Beef Extract 3 g/L, Peptone 5 g/L) and grown using a shaking incubator (26° C., at 200 rpm) overnight.

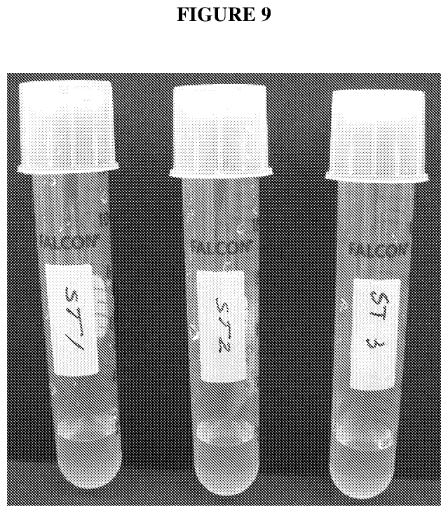

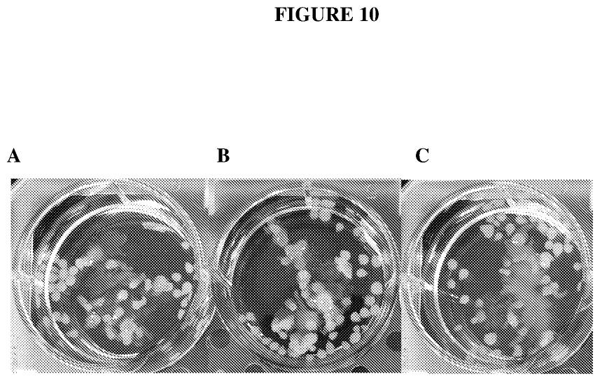

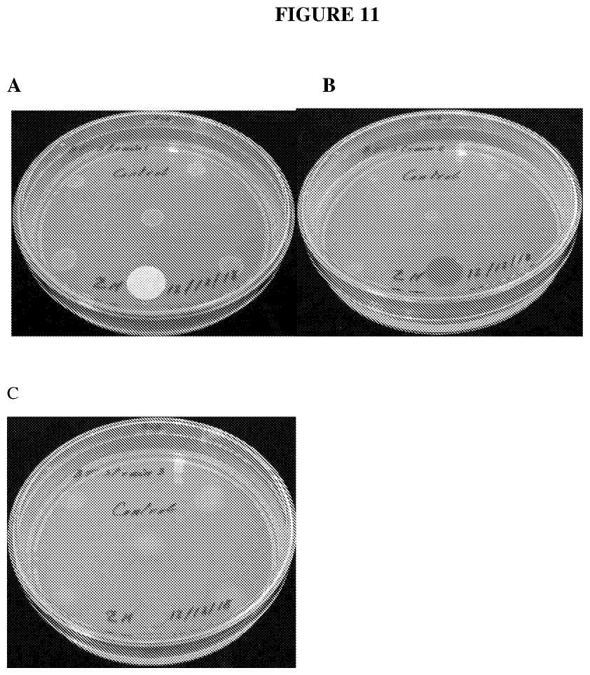

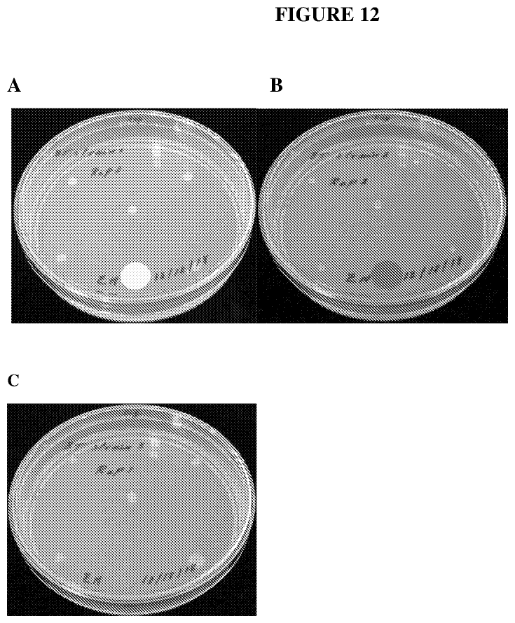

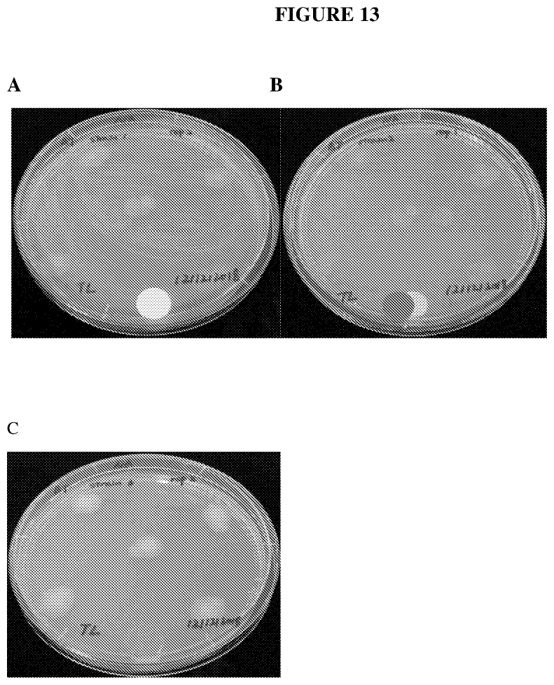

Liquid cultures of each of the microbes in the relevant medium were prepared and 1 ml of each of the microbe suspension culture was mixed with 1 ml 5% sodium alginate medium and briefly mixed in a 14 ml sterile tube (FIG. 9). The solution was then added dropwise c.20-30 μl to 5 ml 50-100 mM CaCl₂.2H₂O solution in a 6 well plate (growth area 95 mm². The culture plate was then incubated at room temperature (22° C.) for 30 minutes. Following this period the CaCl₂.2H₂O solution is discarded as the beads will have formed (FIG. 10). The appropriately formed beads are then manually selected and transferred to a 90×15 mm Petri dish. The 90×15 mm Petri dish contained a range of media, specifically the relevant nutrient broth that the bacteria were initially cultured on as well as the medicinal cannabis regeneration medium (Table 3, shooting rooting medium). In addition the bacterial culture was added directly to the medicinal cannabis regeneration medium to ensure growth on this medium (FIG. 11). The 90×15 mm Petri dishes were cultured at 28° C. for 48 hours, after which the bacteria had completely outgrown the encapsulation on all media, confirming the ability to encase using this method (FIGS. 11-13).

### Example 5

### Encapsulation and Grow-Out of Microbes in Artificial Seeds of Cannabis for Plant Inoculation

In order to inoculate the microbes onto the artificial seeds the methods already described are used with a modification. The procedure explained in Example 1 is followed, to encapsulate the medicinal cannabis bud into an artificial seed. Following this, the procedure explained in Example 4 is performed, with the following exception. Liquid cultures of the microbes are combined with the 5% sodium alginate medium as described above. The artificial seeds that have been created are transferred individually using a wide bore pipette tip that has been cut if necessary, with 20-30 μl of the microbial/sodium alginate mix, ensuring that the artificial seeds are thoroughly coated. Once coated, the artificial seed beads were added to a CaCl₂.2H₂O solution, as described above, to solidify the alginate, thereby encapsulating cannabis bud. Following construction of the microbe-containing artificial seed, regeneration is performed, as described above.

1. An artificial seed comprising isolated meristematic plant tissue encapsulated by a biocompatible polymer, wherein the meristematic plant tissue is derived from a cannabis plant and sterilized prior to encapsulation.

2. The artificial seed of claim 1, wherein the meristematic plant tissue is selected from the group consisting of plant cells, callus tissue, protoplasts, a plant organ, a zygotic embryo and a somatic embryo.

3. The artificial seed of claim 2, wherein the plant organ comprises an adventitious shoot, a micronodule, an axillary bud, an apical bud and/or a scion.

4. The artificial seed of claim 3, wherein the plant organ comprises an axillary bud.

5. The artificial seed of any one of claims 1 to 4, wherein the biocompatible polymer is selected from the group consisting of gelatin, casein, arabinoxylan, soluble starch, chitin, pectin, alginate, methyl cellulose, hydroxyethyl cellulose, methylhydroxypropyl cellulose, methylhydroxyethyl cellulose, hydroxylpropyl cellulose, sucrose and mixtures thereof.

6. The artificial seed of claim 5, wherein the biocompatible polymer comprises alginate.

7. The artificial seed of claim 6, wherein the alginate is sodium alginate or calcium alginate.

8. The artificial seed of any one of claims 1 to 7, wherein the biocompatible polymer comprises a polysaccharide.

9. The artificial seed of claim 8, wherein the polysaccharide is selected from the group consisting of sucrose, lactose and maltose.

10. The artificial seed of claim 9, wherein the polysaccharide is sucrose.

11. The artificial seed of any one of claims 1 to 10, wherein the meristematic plant tissue is encapsulated by at least two layers of biocompatible gel polymers.

12. The artificial seed of any one of claims 1 to 10, wherein the biocompatible polymer comprises an endophyte.

13. The artificial seed of claim 11, wherein at least one of the layers of biocompatible polymers comprises an endophyte.

14. The artificial seed of claim 13, wherein the meristematic plant tissue is encapsulated by an inner layer of a first biocompatible polymer and an outer layer of a second biocompatible polymer, and wherein the inner layer comprises an endophyte.

15. The artificial seed of any one of claims 12 to 14, wherein the endophyte is one or more fungal species.

16. The artificial seed of any one of claims 12 to 14, wherein the endophyte is one or more bacterial species.

17. The artificial seed of any one of claims 12 to 14, wherein the endophyte is one or more fungal species and one or more bacterial species.

18. The artificial seed of claim 16 or claim 17, wherein the one or more bacterial species is selected from the group consisting of Achromobacter, Acidovorax, Acinetobacter, Actinoplanes, Advenella, Aeromicrobium, Agreia, Agrobacterium, Alloprevotella, Anabaena, Anaerococcus, Aquabacterium, Arcicella, Arthrobacter, Averyella, Azospirillum, Bacillus, Bdellovibrio, Beggiatoa, Brachybacterium, Brevundimonas, Bryobacter, Burkholderia, Buttiauxella, Caenimonas, Campylobacter, Chloracidobacterium, Candidatus Microthrix, Castellaniella, Cellulomonas, Cellvibrio, Chryseobacterium, Chthoniobacter, Citrobacter, Clavibacter, Clostridium, Comamonas, Corynebacterium, Coxiella, Cronobacter, Cryocola, Cupriavidus, Curtobacterium, Cytophaga, Dechloromonas, Deinococcus, Delftia, Devosia, Diaminobutyricimonas, Dokdonella, Dongia, Duganella, Enterobacter, Enterococcus, Erwinia, Escherichia, Ferrovibrio, Ferruginibacter, Flavobacterium, Flexibacter, Fluviicola, Frigoribacterium, Fusobacterium, Gaiella, Galbitalea, Gemmata, Gemmatimonas, Geobacter, Giesbergeria, Haliangium, Herbaspirillum, Hirschia, Hydrogenophaga, Inhella, Janthinobacterium, Kineococcus, Klebsiella, Kluyvera, Kosakonia, Kytococcus, Lacibacter, Lactobacillus, Lactococcus, Lautropia, Legionella, Leifsonia, Lelliottia, Leptolyngbya, Leptospira, Leptothrix, Limnohabitans, Luteibacter, Luteimonas, Luteolibacter, Lysinimonas, Lysobacter, Marmoricola, Massilia, Methylobacterium, Methylophilus, Methylotenera, Methyloversatilis, Microbacterium, Micrococcus, Mycobacterium, Neisseria, Nevskia, Niastella, Nitrosomonas, Niveispirillum, Nocardioides, Nostoc, Novosphingobium, Ochrobactrum, Oligoflexus, Opitutus, Oscillatoria, Paenarthrobacter, Paenibacillus, Paludibaculum, Pantoea, Pediococcus, Pedobacter, Peredibacter, Pigmentiphaga, Pirellula, Planctomyces, Prevotella, Propionibacterium, Prosthecobacter, Providencia, Pseudarthrobacter, Pseudohongiella, Pseudomonas, Pseudorhodoferax, Pseudoxanthomonas, Quadrisphaera, Ralstonia, Ramlibacter, Rathayibacter, Reyranella, Rheinheimera, Rhizobium, Rhizomicrobium, Rhodanobacter, Rhodopirellula, Roseiflexus, Roseomonas, Rothia, Rummeliibacillus, Runella, Saccharibacillus, Salinibacterium, Salmonella, Sanguibacter, Segniliparus, Serratia, Shigella, Sodalis, Solirubrobacter, Sphingobacterium, Sphingomonas, Sphingopyxis, Spirosoma, Stenotrophomonas, Stenotrophomonas, Steroidobacter, Streptococcus, Streptomyces, Streptophyta, Tatumella, Thermomonas, Trabulsiella, Trichormus, Tsukamurella, uncultured, Variovorax, Veillonella, Verticia, Wautersiella, Weissella, Xanthomonas, Xylella, Xylophilus and Yonghaparkia.

19. The artificial seed of claim 15 or claim 17, wherein the one or more fungal species is selected from the group consisting of Acremonium, Alternaria, Amorphotheca, Anthracocystis, Apiotrichum, Aplosporella, Apodus, Aspergillus, Aureobasidium, Beauveria, Bipolaris, Candida, Capnodiales, Cercospora, Chaetomium, Chrysosporium, Cladosporium, Clonostachys, Cochliobolus, Coniochaeta, Coniothyrium, Coprinopsis, Corynascella, Cryptococcus, Curvularia, Daldinia, Emericellopsis, Ephelis, Epichloe, Epicoccum, Eurotiales, Exserohilum, Fusarium, Geomyces., Gibberella, Helotiales, Kazachstania, Khuskia, Lecythophora, Leohumicola, Leptosphaerulina, Magnaporthe, Microdiplodia, Microdochium, Microsphaeropsis, Mucor, Muscador, Nodulisporium, Oidiodendron, Ophiosphaerella, Papiliotrema, Paraconiothyrium, Penicillium, Phaeosphaeria, Phaeosphaeriopsis, Phialemonium, Phoma, Pithomyces, Pleosporales., Pseudogymnoascus, Pseudozyma, Pyrenochaetopsis, Ramichloridium, Rhizomucor, Sarocladium, Scopulariopsis, Simplicillium, Sordariales, Sporisorium, Thielavia, Trichosporon, Ustilaginales, Ustilago, Waitea and Xylariales.

20. A method of producing an artificial plant seed of meristematic cannabis plant tissue encapsulated by a biocompatible polymer, the method comprising:
(a) isolating meristematic plant tissue from a cannabis plant;
(b) sterilizing the isolated meristematic plant tissue of (a);
(c) coating the sterilized meristematic plant tissue of (b) in a biocompatible polymer solution; and
(d) exposing the polymer-coated meristematic plant tissue of (c) to a complexing agent to solidify the biocompatible polymer, thereby encapsulating the isolated meristematic cannabis plant tissue with the biocompatible polymer.

21. The method of claim 20, wherein the meristematic plant tissue is selected from the group consisting of plant cells, callus tissue, protoplasts, a plant organ, a zygotic embryo and a somatic embryo.

22. The method of claim 21, wherein the plant organ comprises an adventitious shoot, a micronodule, an axillary bud, an apical bud and/or a scion.

23. The method of claim 22, wherein the plant organ comprises an axillary bud.

24. The method of any one of claims 20 to 23, wherein the biocompatible polymer is selected from the group consisting of gelatin, casein, arabinoxylan, soluble starch, chitin, pectin, alginate, methyl cellulose, hydroxyethyl cellulose, methylhydroxypropyl cellulose, methylhydroxyethyl cellulose, hydroxylpropyl cellulose, sucrose and mixtures thereof.

25. The method of claim 24, wherein the biocompatible polymer comprises alginate.

26. The method of claim 25, wherein the alginate is sodium alginate or calcium alginate.

27. The method of any one of claims 20 to 26, wherein the biocompatible polymer comprises a polysaccharide.

28. The method of claim 27, wherein the polysaccharide is selected from the group consisting of sucrose, lactose and maltose.

29. The method of claim 28, wherein the polysaccharide is sucrose.

30. The method of any one of claims 20 to 29, comprising:
(e) repeating steps (c) and (d) to further encapsulate the meristematic plant tissue in an outer layer of a biocompatible polymer, thereby encapsulating the isolated meristematic plant tissue in an inner layer of a first biocompatible polymer and in an outer layer of a second biocompatible polymer.

31. The method of any one of claims 20 to 29, wherein the biocompatible polymer comprises an endophyte.

32. The method of claim 30, wherein the inner layer of the biocompatible polymer comprises an endophyte.

33. The method of claim 31 or claim 32, wherein the endophyte is one or more fungal species.

34. The method of claim 31 or claim 32, wherein the endophyte is one or more bacterial species.

35. The method of claim 31 or claim 32, wherein the endophyte is one or more fungal species and one or more bacterial species.

36. The method of claim 34 or claim 35, wherein the one or more bacterial species is selected from the group consisting of Achromobacter, Acidovorax, Acinetobacter, Actinoplanes, Advenella, Aeromicrobium, Agreia, Agrobacterium, Alloprevotella, Anabaena, Anaerococcus, Aquabacterium, Arcicella, Arthrobacter, Averyella, Azospirillum, Bacillus, Bdellovibrio, Beggiatoa, Brachybacterium, Brevundimonas, Bryobacter, Burkholderia, Buttiauxella, Caenimonas, Campylobacter, Chloracidobacterium, Candidatus Microthrix, Castellaniella, Cellulomonas, Cellvibrio, Chryseobacterium, Chthoniobacter, Citrobacter, Clavibacter, Clostridium, Comamonas, Corynebacterium, Coxiella, Cronobacter, Cryocola, Cupriavidus, Curtobacterium, Cytophaga, Dechloromonas, Deinococcus, Delftia, Devosia, Diaminobutyricimonas, Dokdonella, Dongia, Duganella, Enterobacter, Enterococcus, Erwinia, Escherichia, Ferrovibrio, Ferruginibacter, Flavobacterium, Flexibacter, Fluviicola, Frigoribacterium, Fusobacterium, Gaiella, Galbitalea, Gemmata, Gemmatimonas, Geobacter, Giesbergeria, Haliangium, Herbaspirillum, Hirschia, Hydrogenophaga, Inhella, Janthinobacterium, Kineococcus, Klebsiella, Kluyvera, Kosakonia, Kytococcus, Lacibacter, Lactobacillus, Lactococcus, Lautropia, Legionella, Leifsonia, Lelliottia, Leptolyngbya, Leptospira, Leptothrix, Limnohabitans, Luteibacter, Luteimonas, Luteolibacter, Lysinimonas, Lysobacter, Marmoricola, Massilia, Methylobacterium, Methylophilus, Methylotenera, Methyloversatilis, Microbacterium, Micrococcus, Mycobacterium, Neisseria, Nevskia, Niastella, Nitrosomonas, Niveispirillum, Nocardioides, Nostoc, Novosphingobium, Ochrobactrum, Oligoflexus, Opitutus, Oscillatoria, Paenarthrobacter, Paenibacillus, Paludibaculum, Pantoea, Pediococcus, Pedobacter, Peredibacter, Pigmentiphaga, Pirellula, Planctomyces, Prevotella, Propionibacterium, Prosthecobacter, Providencia, Pseudarthrobacter, Pseudohongiella, Pseudomonas, Pseudorhodoferax, Pseudoxanthomonas, Quadrisphaera, Ralstonia, Ramlibacter, Rathayibacter, Reyranella, Rheinheimera, Rhizobium, Rhizomicrobium, Rhodanobacter, Rhodopirellula, Roseiflexus, Roseomonas, Rothia, Rummeliibacillus, Runella, Saccharibacillus, Salinibacterium, Salmonella, Sanguibacter, Segniliparus, Serratia, Shigella, Sodalis, Solirubrobacter, Sphingobacterium, Sphingomonas, Sphingopyxis, Spirosoma, Stenotrophomonas, Stenotrophomonas, Steroidobacter, Streptococcus, Streptomyces, Streptophyta, Tatumella, Thermomonas, Trabulsiella, Trichormus, Tsukamurella, uncultured, Variovorax, Veillonella, Verticia, Wautersiella, Weissella, Xanthomonas, Xylella, Xylophilus and Yonghaparkia.

37. The method of claim 33 or claim 35, wherein the one or more fungal species is selected from the group consisting of Acremonium, Alternaria, Amorphotheca, Anthracocystis, Apiotrichum, Aplosporella, Apodus, Aspergillus, Aureobasidium, Beauveria, Bipolaris, Candida, Capnodiales, Cercospora, Chaetomium, Chrysosporium, Cladosporium, Clonostachys, Cochliobolus, Coniochaeta, Coniothyrium, Coprinopsis, Corynascella, Cryptococcus, Curvularia, Daldinia, Emericellopsis, Ephelis, Epichloe, Epicoccum, Eurotiales, Exserohilum, Fusarium, Geomyces., Gibberella, Helotiales, Kazachstania, Khuskia, Lecythophora, Leohumicola, Leptosphaerulina, Magnaporthe, Microdiplodia, Microdochium, Microsphaeropsis, Mucor, Muscador, Nodulisporium, Oidiodendron, Ophiosphaerella, Papiliotrema, Paraconiothyrium, Penicillium, Phaeosphaeria, Phaeosphaeriopsis, Phialemonium, Phoma, Pithomyces, Pleosporales., Pseudogymnoascus, Pseudozyma, Pyrenochaetopsis, Ramichloridium, Rhizomucor, Sarocladium, Scopulariopsis, Simplicillium, Sordariales, Sporisorium, Thielavia, Trichosporon, Ustilaginales, Ustilago, Waitea and Xylariales.

38. An artificial seed produced by the method of any one of claims 20 to 37.
